(19) United States
(12) Patent Application Publication
(10) Pub. No.: US 2005/0255596 A1
(43) Pub. Date: Nov. 17, 2005

(54) Methods of repairing tandemly repeated DNA sequences and extending cell life-span nuclear transfer

(76) Inventors: Michael D. West, Boston, MA (US); Robert P. Lanza, Clinton, MA (US); Jose Cibelli, Holden, MA (US)

Correspondence Address: Joseph Bennett-Paris;MERCHANT & GOULD P.C., P.O. Box 2903, Minneapolis, MN, 55402-0903, US

(21) Appl. No.: 11/079,930

(22) Filed: Mar. 9, 2005

Related U.S. Application Data
(63) Continuation of application No. 09/656,173, filed on Sep. 6, 2000.
Application No. 09/656,173 is a continuation-in-part of application No. 09/527,026, filed on Mar. 16, 2000, now abandoned.
Application No. 09/656,173 is a continuation-in-part of application No. 09/520,879, filed on Apr. 5, 2000, now abandoned.
(60) Provisional application No. 60/152,340, filed on Sep. 7, 1999.
(60) Provisional application No. 60/153,233, filed on Sep. 13, 1999.
(60) Provisional application No. 60/179,486, filed on Feb. 1, 2000.

(51) Int. Cl. C12Q 1/68; C12N 15/63; C12N 5/08; C12N 15/85

(52) U.S. Cl. 435/455; 435/366

(57) ABSTRACT

This invention relates to methods for rejuvenating normal somatic cells and for making normal somatic cells of a different type having the same genotype as a normal somatic cell of interest. These cells have particular application in cell and tissue transplantation. Also encompassed are methods of re-cloning cloned animals, particularly methods where the offspring of cloned mammals are designed to be genetically altered in comparison to their cloned parent, e.g., that are “hyper-young.” These animals should be healthier and possess desirable properties relative to their cloned parent. Also included are methods for activating endogenous telomerase, EPC-1 activity, and or the ALT pathway and/or extending the life-span of a normal somatic cell, and other genes associated with cell aging and proliferation capacity.

### CROSS-REFERENCE TO RELATED APPLICATION

This application is a continuation-in-part of U.S. Ser. Nos. 09/527,026 and 09/520,879, and claims benefit of provisional application 60/152,340 and 60/153,233.

### FIELD OF INVENTION

The present invention relates to methods for rejuvenating normal or modified somatic cells or cellular DNA that is senescent, checkpoint arrested, nearing senescence or has an undesirably short cell life, through nuclear transfer techniques. The methods are particularly useful for rejuvenating cells which have reached or are approaching senescence due to clonal expansion following complex genetic manipulations or from tissue chronic tissue injury, and thereby increase the potential of such cells to serve as donors for the generation of cloned transgenic animals or for cell therapy in humans.

Also the invention is useful for rejuvenation of cells which are senescent or aged as a result of chronologic aging or because of conditions associated with exacerbated cell senescence such as muscular dystrophy or atherosclerosis, imnumosenescence, BPH, neurodegenerative diseases, Barrett esophagus cirrhosis, AMD osteoarthritis and skin ulcers. The patient or animal's cells will be reprogrammed or rejuvenated by nuclear transfer or related technique and regenerated and restored to totipotency. These totipotent cells may be used to produce cell types including but limited to pluripotent cells such as mesenchymal or premesenchymal stems cells, hematopoietic cells, vascular cells and so on, which can be transplanted into the patient or animal or suitable donor. These cells will “seed” the patient or animal's tissues with healthy proliferation competent cells of numerous types including bone, blood, muscle, neurons, immune cells, and other types.

The methods of the invention also include the making of differentiated cells from rejuvenated cells, and teratomas which contain cells from any or all three germ layers and are useful for making primary cells of a different type having the same genotype as a primary cell of interest. Such newly generated primary cells have important significance in the field of tissue engineering and organ replacement therapy. Also encompassed are methods of re-cloning cloned mammals, particularly methods where the offspring of cloned mammals are designed to be genetically altered in comparison to their cloned parent.

Also the invention relates to assays for identifying compounds that moderate cell aging and senescence, and genes associated therewith, in particular compounds that affect telomere length, EPC-1 activity, tPA, collagenase activity, gas genes, mitotic index, and other indications of cellular aging and proliferation capacity.

### BACKGROUND OF THE INVENTION

The past decade has been characterized by significant advances in the science of cloning, and has witnessed the birth of a cloned sheep, i.e. “Dolly” (Roslin Bio-Med), a trio of cloned goats named “Mira” (Genzyme Transgenics using technology licensed from ACT), several dozen cloned cattle (ACT), numerous generations of cloned mice, and very recently, five cloned pigs (PPL). The technology which enables cloning has also advanced such that a mammal may now be cloned using the nucleus from an adult, differentiated cell, which scientists now know undergoes “reprogramming” when it is introduced into an enucleated oocyte. See U.S. Pat. No. 5,945,577, herein incorporated by reference.

The fact that an embryo and embryonic stem cells may be generated using the nucleus from an adult differentiated cell has significant implications for the fields of organ, cell and tissue transplantation. For instance, embryonic stem cells generated from the nucleus of a cell taken from a patient in need of a transplant could be made, and induced to differentiate into the cell type required in the transplant. By using techniques evolving in the field of tissue engineering, tissues and organs could be designed from the cloned differentiated cells which could be used for transplantation. Because the cells and tissues used for the transplant would have the same nuclear genotype as the patient, the problems of transplant rejection and the dangers inherent in the use of immune-suppressive drugs would be avoided or decreased. Moreover, the engineered cells and tissues could be readily modified with heterologous DNA, or modified such that deleterious genes are inactivated, such that the transplanted cells and tissues are genetically corrected or improved if necessary. US Application Serial No. ______, co-owned and filed concurrently with the present invention, discusses methods for genetically modifying both the donor nuclear DNA and the recipient mitochondrial DNA, and is herein incorporated by reference in its entirety.

There have been recent concerns, however, regarding the genetic age of cloned cells. A recent report by Shiels et al. (Nature (1999) 399: 316), involving Dolly, the cloned sheep, suggests that nuclear transfer may not restore telomeric length, and that the terminal restriction fragment (TRF) size observed in animals cloned from embryonic, fetal and adult cells reflects the mortality of the transferred nucleus. The implications of these findings are particularly relevant for the cloning of replacement cells and tissues for human transplantation (Lanza et al. (1999a) Nature Med. 5: 975; Lanza et al. (1999b) Nature Biotechnol. 17: 1171). Transplanted organs which undergo premature senescence could become destructive to surrounding tissue in vivo and could actually aggravate the disease which the replacement cells are intended to treat. The Shiels et al. report also raises questions as to whether cells created by nuclear transfer will undergo premature senescence and whether cloned animals generated by nuclear transfer will exhibit decreased life spans. This in turn has serious implications for the cloning and re-cloning of high quality farm animals, which, prior to the report, was considered to be advantageous over traditional breeding techniques which are dependent on the animals reaching mating age before another generation may be propagated.

Scientists have hypothesized that telomere loss is linked to the aging process for at least two decades. See, Harley, “Telomere loss: mitotic clock or genetic time bomb?” Mutation Res. (1991) 256: 271-282. The hypothesis, originally called the “marginotomy theory,” is that the gradual loss of chromosomal ends, or telomeres, leads to cell cycle exit and as a consequence, cell senescence. See Olovnikov, “A theory of Marginotomy” J. Theor. Biol. (1973) 41: 181-190. The hypothesis originally arose through the prediction that DNA polymerase, because it required an RNA primer for the replication of the lagging stand, would be unable to completely replicate the ends of chromosomes. This prediction was eventually confirmed through molecular studies which showed that the mean length of terminal restriction fragments in human fibroblast chromosomes were decreased in a replication dependent manner in vitro. See Harley et al. “Telomeres shorten during aging of human fibroblasts”, Nature (1990) 345:458-460.

Further evidence supporting the telomere theory relates to the enzyme telomerase. Telomerase activity in human cells was first identified in 1989. See Morin, “The human telomere terminal transferase is a ribonucleoprotein that synthesizes TTAGGG repeats” Cell (1989) 59: 521-529. Telomerase acts to build on the ends of chromosomes, restoring telomere length. Other studies have shown that, while telomerase activity is repressed during differentiation of somatic cells, telomerase is active at some stage of germ-line cell replication and thus maintains telomere length in germ cells between generations. In addition, telomerase has also been shown to be active in transformed cells. See Harley 1991) for a review.

It has been proposed that the suppression of telomerase in differentiated cells may function to limit the capacity of somatic cells to clonally expand in an uncontrolled manner, as in cancer. But some tumor cell lines show a telomerase negative immortality that has been designated the ‘ALT’ pathway. The inventors propose that this alternative pathway, like the acquisition of telomerase activity in tomorigenesis, is the reappearance of a germ-line trait. The inventors propose that damaged telomeres are repaired in the germ line, not only through the addition of telomeric repeats by telomerase, but also through homologous strand invasion and extension by DNA polymerase.

[image]

Because nuclear transfer bypasses sexual reproduction, i.e., uses a somatic as opposed to a germ cell as the source of nuclear DNA, a current hypothesis with regard to cloning is that the telomeres of clones are never regenerated, and that a cloned animal is of the same “genetic age” as its parent. In fact, it has even been noted that the technology involved in cloning further reduces the length of telomeres, because cells are cultured in the laboratory for a period of time before being used for nuclear transfer. See BBC News, “Is Dolly old before her time?” Thurs., May 27, 1999. If this theory were true, it would mean that cells from clones may have a much shorter average life span than those from an animal of the same age generated via sexual reproduction, and perhaps the animals may have a shorter life span than the parents from which they are generated.

Not only does the this theory have serious implications for the field of organ transplantation, but it also calls into question the extent of genetic manipulations which may be performed to somatic cells which are to be used for nuclear transfer. For instance, a major advantage of nuclear transfer technology is that somatic cells may be more readily maintained in culture and transfected with transgenes than embryonic stem cells. This property facilitates the production of animals which produce therapeutic proteins, i.e., for instance cows which express transgenes from mammary-specific promoters enabling the production of therapeutic proteins in milk. Likewise, if cells used for nuclear transfer were not able to undergo a series of genetic manipulations because of aging chromosomes, it would be virtually impossible to generate animals, cells and tissues with multiple genetic manipulations. The ability to perform such complex genetic manipulations, however, may be necessary, for example, to correct genetic abnormalities in donor cells from patients having deleterious mutations before such cells are used for nuclear transfer and organ transplantation.

One hypothesis to explain why some researchers have observed that telomeres were not regenerated following nuclear transfer is that telomere regeneration will be dependent on the choice of donor somatic cell types. Recent studies have shown that reconstruction of telomerase activity leads to telomere elongation and immortalization of normal human fibroblasts and retinal epithelial cells (Bodnar et al. (1998) Science 279: 349; Vaziri and Benchimol (1998) Curr. Biol. 8: 279), whereas similar experiments using mammary epithelial cells did not result in elongation of telomeres and extended replicative life span (Kiyono et al. (1998) Nature 396: 84). Differences between cells in the ability of telomerase to extend telomeres, or in the signaling pathways activated upon adaptation to culture, were proposed to explain the differences (de Lange and DePinho (1999) Science 283: 947).

Some researchers have suggested that telomerase activity may be cell-cycle dependent. For instance, in 1996, Dionne reported the down-regulation of telomerase activity in telomerase-competent cells during quiescent periods (G. phases) and hypothesized that telomerase activity may be cell-cycle dependent. See http://telomeres,virtualave.net/regulation.html. Similarly, Kruk et al. reported a higher level of telomerase in the early S phase when compared to other points in the cell cycle (Biochem. Biophys. Res. Commun. (1997) 233: 717-722). However, other researchers have reported conflicting results, and have alternatively suggested that telomerase activity correlates with growth rate, not cell cycle (Holt et al. (1996) Mol. Cell. Biol. 16(6): 2932-2939; see also Website, id., referencing Holt, 1997, and Belair, 1997). Still others have proposed that telomerase activation is mediated by other cellular activation signals, as evidenced by the upregulation of telomerase in B cells in vitro in response to CD4O antibody/antigen receptor binding and exposure to interleukin-4 (Website, id., citing Weng, 1997; see also Hiyama et al. (1995) J. Immunol. 155 (8): 3711-3715). But despite the rising interest in telomerase and its purported role in the process of aging and cellular transformation, the regulation of telomemse activity remains poorly understood. See, e.g., Smaglik, “Turning to Telomerase: As Antisense Strategies Emerge, Basic Questions Persist,” The Scientist, Jan. 18, 1999, 13(2): 8).

The ability to regulate telomerase activity could have wide-reaching effects in the medical community, and has the potential to profoundly influence many more technologies than the regeneration of telomeres in cloned animals. Having the ability to regulate telomerase will enable the treatment of many age-related and other types of disease processes. For instance, the capability to regulate telomerase could be important for improving the effectiveness of bone marrow transplants in connection with cancer chemotherapy; telomerase therapy may be useful in replacing age-worn cells in the immune system, and in the retina of the eye for example, in treating the lining of blood vessels to help prevent heart attack or stroke, extending the life span of hepatocytes for the treatment of cirrhosis, or myoblasts in muscular dystrophy. Moreover, the capability to regulate telomerase may permit the control of cancerous cells. Finally, an in vitro model of telomere and telomerase regulation, in particular, a model for the reversal of cellular aging, would enable the design of assays and screens to identify the molecular mechanisms of telomere regulation, aging, and cancer. Thus, a better understanding of the regulation of telomerase has the potential to lead to a wide range of treatments, in addition to securing the efficacy of cloned tissues for tissue engineering and transplants, and ensuring and even increasing the life span of cloned and non-cloned animals.

### SUMMARY OF THE INVENTION

The present invention is based on the surprising discovery, in light of the recent doubts about the genetic age of cloned mammals, that the process of nuclear transfer is capable of rejuvenating senescent or near-senescent cells and repairing tandemly repeating DNA sequence such as that in the telomeres, restoring youthful patterns of gene expression such as increasing EPC-1 activity, and/or increases cell life span or cell proliferation capacity. The present invention therefore enables what would not have been deemed possible in light of the recent concerns about nuclear transfer; namely, that cells that are at or near senescence, e.g., those grown in culture until they are near senescence, or obtained from humans or animals having age-related defects or conditions may still be used to generate cloned cells, tissues and animals having telomeres that are at least comparable in length, or longer, than age-matched controls. Also, these cells possess patterns of gene expression of young cells, such as increased EPC-1 activity relation to donor cells. Moreover, the present invention establishes, in contrast to what had been recently suggested, that generating clones of clones, i.e. “re-cloning,” is entirely feasible, and may be repeated theoretically indefinitely, thereby resulting in “hyper-young” cells, tissues, organs and animals.

Telomere shortening is currently believed to lead to chromosome ends that are indistinguishable from double strands breaks thereby signaling DNA damage checkpoint (W. E. Wright & J. S. Shay 2000, Nat. Med. 6(8) 849-851.)

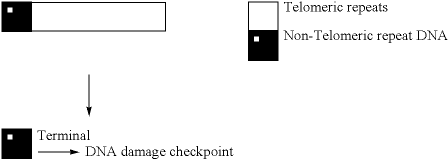

Telomeres may, however, contain an increasing amount of degenerate or non-telomeric repeat DNA progressing centromeric from the telomere.

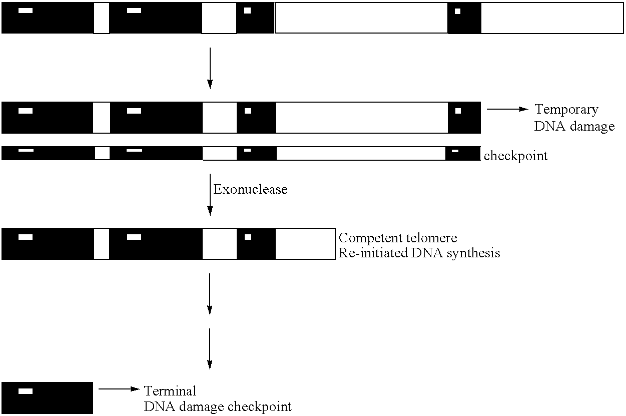

The appearance of these non-telomeric repeat sequences causes a temporary DNA damage checkpoint. Following repair, such as though exonuclease activity, the cell can re-enter the cell cycle. The growth of a mortal cell to terminal senescence with subsequent nuclear transfer causes the synthesis of an extended array of uniform telomeric repeat sequences that do not always appear in nature. 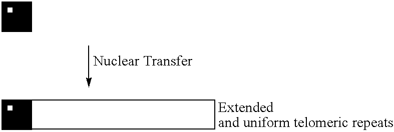
Cells and/or animals containing chromosomes with such extended and uniform telomeric repeat sequences will be rejuvenated, and have the unique characteristic of being hyper-young, as a mass population of cells having fewer cells in DNA damage checkpoint at any one period of time.

The present invention is based on the discovery that nuclear transfer techniques may be used to extend the life span of somatic cells, e.g., senescent or near-senescent or checkpoint arrested cells by activating endogenous (cellular) telomerase activity, and young patterns of gene expression by the repair of tandemly repeated DNA sequence damage. This provides particular advantages over recently suggested approaches for resolving the telomere loss seen in nuclear-transfer generated animals, which focus on the exogenous expression of a cloned telomerase gene to resolve telomere shortening in cloned mammals.

In this regard, researchers at Geron Corporation and the Roslin Institute have recently collaborated to combine Geron's cloned telomerase gene (hTERT) with nuclear transfer in order resolve telomere shortening in clones. See, e.g., Business Wire, May 26, 1999. This announcement preceded the May 27th Nature report by researchers at Roslin Institute that two other sheep (after Dolly) cloned by nuclear transfer also exhibit shorter telomeres than age-matched controls. Researchers at the University of Massachusetts involved in cloning cattle also believed that transfecting donor cells with an exogenous telomerase gene might be beneficial for the life-span of cloned animals, despite their observation that nuclear transfer seemed to rejuvenate senescent donor cells. See http://abcnews.go.com/sections/science/Daily News/clones980522.html (1998).

The present invention is advantageous over proposed methods to express telomerase from a transfected telomerase gene, in that no genetic manipulations are required to activate telomerase and regenerate telomere length in cloned cells, tissues and animals. In addition, the up-regulation of telomerase activity achieved with the present invention is transient, and while it is sufficient to extend telomere length, it does not impart constitutive immortality. This advantage is particularly significant given the observation that telomerase is constitutively upregulated in many types of cancer cells and constitutive telomerase expression has been reported to result in the up—regulation of the proto-oncogene c-MYC (D. Bead paper). Therefore, introducing an extra gene for telomerase also introduces the possibility of inducing cell transformation, and will likely require subsequent measures aimed at controlling telomerase expression from the transfected gene. A method whereby telomerase activity may be controlled using the cell's own regulatory mechanisms is therefore preferable to inserting exogenous copies of the telomerase gene.

In addition, the present invention is advantageous over the exogenous expression of telomerase in that the culture of somatic cells leading to telomere shortening with subsequent nuclear transfer to extend telomeres results in a population of rejuvenated cells all of which have more uniform tracts of telomeric repeats. As a result individual cells isolated from such a population have a greater probability of being competent for extended proliferation and the population will have the unique property of having fewer checkpoint arrested cells than natural cells, thereby being “hyper young.”

Thus, encompassed in the invention are methods of rejuvenating or increasing the life-span of normal somatic cells using nuclear transfer. The somatic cells which would benefit from the disclosed methods include any somatic cell, e.g. a cell which is nearing senescence, either by reaching the natural limit on population doublings or as a result of harsh selection conditions for complex genetic alterations or conditions, that have exposed the cell to high oxygen tension or other conditions that have damaged telomeric DNA. As discussed, this includes especially cells from patients or animals with age related deficiencies or conditions such as age related macular degeneration, immune senescence neurodegenerative disorders such as Parkinson's or Alzheimer's diseases, osteoarthritis, muscular dystrophy, skin aging, emphysema, aneunsnis, coronary heart disease, atherosclerosis, hypertension, cataracts, adult onset diabetes. Also, the invention has application in conditions associated with accelerated cell turnover such as muscular dystrophy, herpes zoster, AIDS, and cirrhosis. The present methods are applicable to any somatic cell of interest, and use of such cells as donors for nuclear transfer.

The methods of the invention allow one to reprogram the nucleus of a late passage somatic cell to an embryonic state. By allowing the embryonic cell to differentiate and develop into many different cell types, one may re-isolate the primary cell of interest in a rejuvenated or “young” state. Also, since the methods of the invention entail making an embryonic stem cell which differentiates into all different cell types, any type of cell may be generated using any primary cell of interest, so long as the genome of the somatic cell has not been altered as to affect cellular development. Thus, the invention provides an invaluable way to analyze the affect of the same genetic alteration in an isogenic background (i.e., a gene knock-out or expression of a heterologous gene) in different cell types in vitro.

For example, a patient's somatic cells may be reprogrammed by a NT related technique, regenerated and restored to totipotency. From these rejuvenated totipotent cells; pluripotent stem cells can be obtained such as pre-mesenchymal, mesenchymal, enangioblasts, hematopoietic stem cells. These pluripotent cells or cells derived therefrom can be transplanted into donors where they will “seed” the patient's tissues with healthy proliferation competent cells, such as immune cells, blood cells, bone, muscle, neural, and other types.

The methods of the present invention also increase the life-span of a desired cell, preferably a mammalian cell, and more preferably is a human cell, e.g., that is in need of rejuvenation, by using said cell, the nucleus or chromosomes therefrom, as a nuclear transfer donor. Preferably the process will be repeated, in that cells, nuclei or chromosomes obtained from the resultant cloned embryo will themselves be used as nuclear transfer donors. Also, the donor cells will preferably be transgenic.

The methods of the present invention further allow one to restore repetitive DNAs in desired cells and to activate or modulate (reduce or increase expression) those genes involved in aging including telomerase in desired cells, e.g., mammalian cells in need of rejuvenation, and checkpoint arrested cells, by using said cell, the nucleus or the chromosomes derived therefrom as a donor during nuclear transfer or exposing the DNA of such cell to an embryonic cell type. As discussed in detail infra, this is an unparalleled discovery as the present invention may provide a means for identifying specific molecules that are involved in the aging of cells, and which regulate cell life-span. Specifically, the invention provides assays to identify compounds that restore repetitive DNAs such as telomeres, activate or inhibit genes altered in the course of cell aging such as telomerase, gas, tPA and others.

In view of the inventor's finding that nuclear transfer may be used to rejuvenate or increase the life-span of mammalian cells, e.g. cells at or near senescence, it is no longer a concern that cloned mammals, fetuses, teratomas, or embryos, or inner cell masses or blastocysts are of the genetic age of their parents. Thus, the invention also encompasses methods of re-cloning cloned mammals, fetuses, teratomas, embryos, etc. using nuclear transfer techniques. Such re-cloning methods are particularly useful for making transgenic mammals expressing more than one heterologous gene, or having more than one gene knocked out, because such animals can be generated by cloning techniques to generate cloned and re-cloned mammals of the same genetic background. Such methods forego the need for mating or breeding, which often results in other genetic differences and may be impossible for obtaining double knockout or double transgenic mammals having altered genes which are closely linked on the genome such that they are inherited together.

### BRIEF DESCRIPTION OF THE DRAWINGS

FIG. 1. Characterization of cell senescence in NT donor cells. (A) Cells were observed by phase contrast microscopy. The donor cells displayed an increased cell size and cytoplasmic granularity (b) as compared to the early passage BFF cells (a). (B) Representative electron micrographs of BEF (a) and donor CL53 (b) cells. Note the convoluted nucleus (n) of CL53 cells. CL53 cells are larger than BFF cells, and their cytoplasm contain abundant lysosomes (arrows) and thick fibrils. Both pictures are at the same magnification. The bar represents 2 microns. Mitochondria (in). (C) Entry of early (a, BFF) and late passage (b, CL53) cells into DNA synthesis as determined by ³H-thymidine incorporation during a 30 hr incubation (V. J. Cristofalo and B. B. Sharf (1973) Exp. Cell Res. 76:419). The cells were processed for autoradiography, and then observed microscopically and scored for labeled nuclei. At least 400 nuclei were counted to determine the percentage of labeled nuclei, following an established protocol (Cristofalo and Sharf (1973)). (D) The donor CL53 cells exhibit reduced EPC-1 mRNA levels as determined by Northern analysis. Human fibroblasts (WI-38) at early passage (Y) and late passage (O), bovine fibroblasts at early passage (Y; BFF) and late passage (0; donor CL53), and RNAs isolated from cloned calf dermal fibroblast strains are indicated. RNA was extracted from the cells after they were grown to confluence and growth-arrested in serum free medium for 3 days (P. Chomczynski and N. Sacchi (1987) Anal. Biochem 162: 156). Equal amounts of RNA were treated with glyoxal, separated by electrophoresis on agarose gels, transferred to nitrocellulose filters electrophoretically, and hybridized with the full length EPC-1 cDNA using standard conditions (D. G. Phinney, C. L. Keiper, M. K. Francis, K. Ryder (1994) Oncogene 9: 2353).

FIG. 2. Normal cows cloned from senescent somatic cells. (A) CLS3-8, CL53-20 9, CL53-10, CL53-11 and CL53-12 (nicknamed Lily, Daffodil, Crocus, Forsythia, and Rose, respectively) at 5 months of age; and (B) CL53-1 (Persephone, insert) at 10 months of age.

FIG. 3. Ability of nuclear transfer to restore the proliferative life-span of senescent donor cells. (A) The growth curve of the original BFF cell strain (*) is compared to that of cells derived from fetus (ACT99-002) (o) that was cloned from late passage BFF cells (CL53 cells). (B) The growth curve of the CL53 donor cells demonstrating that the cultures bad approximately 2 population doublings remaining. (C) Late passage CL53 cells (n=97) were seeded at clonal density, and the proliferative capacity after 1 month was collated. (D) In contrast to the clones derived from late-passage cells, single cell clones from early passage BFF cultures and early-passage ACT99-002 (clone) showed a capacity for extended proliferation.

FIG. 4. Telomere length analysis. (A) Nucleated blood cells. Peripheral blood samples from cloned and control animals were analyzed by flow FISH(N. Rufer, W. Dragowska, G. Thombury, E. Roosnek, P. M. Lansdorp (1998) Nature Biotechnol. 16: 743) in two separate blinded experiments. Duplicate samples of nucleated cells (pooled granulocytes and lymphocytes) obtained after osmotic lysis of red cells using animonium chloride were analyzed by flow FISH as described (N. Rufer et al. (1999) J. Exp. Med. 190: 157). The average telomere fluorescence of gated mononuclear cells was calculated by subtracting the mean background fluorescence from the mean fluorescence obtained with the FITC-labeled telomere probe. Note that the age-related decline in telomere fluorescence values in normal cows and the relatively long telomeres in the cloned animals. (B) Analysis of terminal restriction fragments. Genomic DNA isolated from control cells (pre-transfection BFF bovine fibroblasts), senescent CL53 cells and fibroblasts from a 7 week old cloned fetus (ACT99-002) cells obtained by NT with senescent CL53 cells. TRF analysis of DNA fragments obtained following digestion with Hinfl/RsaI was performed on a 0.5% agarose gel run for 12 hours as described (Telomere Length Assay Kit, Pharmingen, San Diego, Calif.). Lane 1: controls DNA from CEPH lymphoblastoid human cell line 134105; lane 2: biotinylated markers (Pharmingen); lane 3: TeloLow control DNA (Pharmingen, mean TRF length 3.3 kb); lane 4: senescent CL53 cells; lane 5: BFF fibroblasts pre-transfection; lane 6: ACT99-002 (cloned) cells. (C) TRE analysis as in B following electrophoresis for 24 hours on a 0.5% agarose gel Lane 1: ACT99-002 cells (mean TRF length 19.3 kb); lane 2: BFF056H fibroblasts pre transfection (mean TRF length 17.9 kb); lane 3: senescent CL53 cells (mean TRF length 16.2 kb); lane 4 TeloHigh control DNA (Pharmingen, mean TRF length 11.3 kb); lane 5: control DNA from CEPH lymphoblastoid human cell line 134105; lane 6 biotinylated lambda DNA cut with Hind III (molecular weight markers). (D) Flow FISH analysis of pre-transfection BIT bovine fibroblasts, senescent CL53 cells and ACT99-002 fibroblasts. Cells were analyzed following hybridization with or without FITC-©3TA2)₃ peptide nucleic acid probe (respectively gray and black histograms). Single cells were gated on the basis of light scatter properties. Note the higher autofluorescence in the senescent CLS3 cells used as nuclear donor. Fluorescence was measured on a linear scale. After subtraction of background fluorescence ACT99-002 (cloned) cells have the highest fluorescence followed by BFF cell. The senescent CL53 cells appear to have the lowest specific fluorescence.

FIG. 5. Telomerase is expressed in reconstructed embryos but not in donor bovine fibroblasts. Telomerase activity was measured using a Telomeric Repeat Amplification Protocol (TRAP) assay kit (Pharmingen, San Diego, Calif.). Lysates from adult donor senescent (CL53) fibroblasts and day 7 reconstructed bovine embryos (n=15) were obtained and used in the TRAP assay. Lane 1: extract from 4000 K562 human erytbroleukemia cell lie cells; lane 2: 20 bp ladder; lane 3: no cell extract; lane 4: heat treated embryo (n=1) extract; lane 5, n=10; lane 6, n=1; lane 7, n=0.1; lane 8, n=0.01); lane 9 extract from 4000 donor CL53 fibroblasts; lane 10-11 controls for fibroblast extract (resp. no TS template and heat inactivated extract); lane 12: 20 bp ladder. All lanes contain the internal control TRAP reaction (36 bp).

### DETAILED DESCRIPTION OF THE INVENTION

The present invention includes methods of rejuvenating normal somatic cells.

“Normal somatic” cells is intended to mean that such cells that are committed to a somatic cell lineage are not tumorgenic or transformed, and are capable of being reprogrammed and of facilitating embryonic development after said cell or a nucleus of such a cell or chromosome from said cell is transferred to an enucleated oocyte or otherwise exposed to factors present in germ line cells. Normal somatic cells may or may not be genetically modified. By “rejuvenated” the inventors mean at least one of the following: that the possible number of population doublings remaining for said somatic cell is increased, that EPC-1 activity or other markers of cellular aging are reversed to a youthful state; that telomerase is upregulated, and/or that telomeres are increased “Hyper-young” indicates that the population of cells have markers of cellular aging that are younger than normal cells.

“Terotoma” refers to a group of differentiated cells containing derivatives of mesoderm, endoderm, or ectoderm resulting from totipotent cells.

In a preferred embodiment of the invention, the normal somatic cells to be used for the present invention are senescent cells, checkpoint arrested cells, or cells that are near-senescence. However, the present methods are applicable for any desired normal somatic cell, preferably a human cell. Replicative senescence is a physiological state distinguishable from quiescence achieved by either serum starvation or density-dependent inhibition of growth of young cells (West et al. (1989) Exp. Cell Res. 184: 138; West et al. (1996) Exp. Gerontol. 31: 175; and Pignolo et al. (1998) Exp. Gerontol. 33: 67), and appears to involve a block in late G₁ near the G₁/S boundary in the cell cycle (Cristofalo and Pignolo Exp. (1996) Gerontol. 31: 111; Gorman and Cristofalo (1986) Exp. Cell Res. 167: 87; and Cristofalo et al. (1992) Aging and Cellular Defense Mechanisms, Franceshi et al., Eds. (New York Academy of Sciences, New York), pp. 187-194).

Senescent cells may be identified by a variety of means known in the art. For 15 instance, phase contrast light microscopy, and ultrastructural analysis by electron microscopy may be used to verify features of fibroblast replicative senescence, including prominent and active Golgi apparati, increased invaginated and lobed nuclei, large lysosomal bodies, and an increase in cytoplasmic microfibrils as compared to the young cells (Lipetz and Cristofalo (1972) J. Ultrastruct. Res. 39: 43). In addition, senescent cells have a reduced capacity to enter S phase as measured by a decrease in the incorporation of ³H-thymidine and a significant increase in the staining of senescence-associated β-galactosidase (G. P. Dirni et al (1995) Proc. Natl. Acad. Sci. USA 92: 9363). Senescent cells also exhibit a reduction in EPC-1 (early population doubling level cDNA-1) (Pignolo et al. (1993) J. Biol. Chem. 268: 8949) mRNA levels as compared to early passage cells, and a down-regulation of gasI gene expression as compared to quiescent cells (Cowled et al. (1994) Exp. Cell Res. 211: 197-202).

Senescent cells can be isolated by propagating cells until they reach a state of irreversible growth arrest. By “near-senescence” the present inventors mean that such cells have the capability to divide no more than about three to six times, but are preferably less than two or three population doublings from replicative senescence. Although the preferred means of generating senescent cells for nuclear transfer is to passage normal somatic cells until greater than about 90 to 95% of their life-span is completed, senescence and senescent-like states can also be induced by exposing cells to various agents, including genotoxic agents and Cdk inhibitors (McConnell et al. (1998) Current Biol. 8: 351-354). Genotoxic agents induce a growth arrest similar to senescence and distinct from quiescence called DNA damage checkpoint arrest. Alternatively, near-senescent cells can be obtained from animals or humans, e.g., those with aging associated conditions.

The methods of the present invention may employ cell rejuvenation to generate cloned animals, or may be used to rejuvenate a normal somatic cell of interest for other purposes. Such methods may include:
a. transferring said somatic cell, the nucleus from said somatic cell, or chromosomes from said somatic cell to a recipient oocyte or egg or other suitable recipient cell in order to generate an embryo;
b. obtaining an embryo having at least one cell, an inner cell mass, embryonic disc and/or stem cell using said embryo;
c. allowing said embryo, inner cell mass, embryonic disc and/or stem cell to differentiate into desired cell or tissue types;
d. isolating said resulting cells or tissues;
e. transplanting said cells or tissues into patient.

The differentiated cells teratomas, inner cell masses, embryonic disc and embryonic stem cells isolated according to the invention will have telomeres that are at least as long if not longer than those of the donor normal somatic cell, and are also an aspect of the invention. Also, these differentiated cells should possess markers of cellular aging that are young or hyper-young. A method whereby the differentiated cells or tissues, teratoma cells, inner mass cells, blastocyst cells or embryonic cells are then used as subsequent nuclear donors is also envisioned. Such a method is particular suitable for isolating normal somatic cells, teratomas, ES cells, etc. having multiple transgenes or genetic alterations, and may be repeated indefinitely until the desired number of genetic changes have been accomplished.

The normal somatic cell used for the methods of the invention may be any cell 20 type. Suitable cells include by way of example immune cells such as B cells, T cells, dendritic cells, skin cells such as keratinocytes, epithelial cells, chondrocytes, cumulus cells, neural cells, cardiac cells, esophageal cells, dermal fibroblasts, cells of various organs including the liver, stomach, intestines, lung, pancrease, cornea, skin, gallbladder, ovary, testes, other reproduction organs, kidneys, etc. In general, the most appropriate cells are easily propagatable in tissue culture and can be easily transfected. Preferably, cell types for transfecting heterologous DNA and performing nuclear transfer are fibroblasts.

Methods and protocols for effecting nuclear transfer are disclosed in U.S. Pat. No. 5,945,577; U.S. Ser. No. 08/888,057, filed Jul. 3, 1997; U.S. Ser. No. 08/888,283, filed Jul. 3, 1997; U.S. Ser. No. 08/935,052, filed Sep. 22, 1997; and U.S. Ser. No. 09/394,902, filed Sep. 13, 1999, all of which patent and applications are incorporated by reference in their entirety herein.

The somatic cell may be from any type of animal or mammal, such as pig, goat, cat, dog, rat, mouse, bovine, buffalo, sheep, horse, human, non-human primate, but is preferably an ungulate cell, and most preferably a bovine cell. The oocyte or egg used for nuclear transfer will be from similar sources and can be of the same or different species than donor cell or DNA.

The immune-compromised animal may be any animal capable of supporting teratoma formation, and is immune-compromised to the extent that no rejection of the developing teratoma occurs. For example, the immune-compromised animal may be a SCID or nude mouse. Alternatively, cells may be differentiated in vivo or in avian eggs.

The method is particularly useful for isolating somatic cells having complex or compound manipulations, i.e., more than one transfected heterologous gene and/or gene knockout, where it may be difficult to keep the somatic cell in culture long enough to affect all the desirable genetic alterations. First, somatic cells, preferably those made hyper-young by nuclear transfer, are used as a substrate for gene targeting. Thus, the somatic cell could undergo a first genetic manipulation, could then be rejuvenated according to the methods of the invention, and could then go through a second genetic manipulation once the genetic clock has been “reset.” Accordingly, a rejuvenated somatic cell according to the invention may have at least one alteration to the genome depending on the complexity of the genetic manipulation and the number of times it has gone through the rejuvenation process. Rejuvenated, genetically altered cells generated by the methods of the invention are also encompassed.

The invention also includes methods of making somatic cells having the same genotype as a first cell which is of a different cell type. Such a method is made possible by the process of rejuvenation, which is effected by transferring a first somatic cell, the nucleus of a first primary cell, or the chromosomes from a first primary cell into an enucleated recipient oocyte or other suitable recipient cells, or by contacting the somatic cell with proteins in the oocyte to generate a teratoma or other mass of differentiated cells, which contains derivatives of any of the germ layers ectoderm, mesoderm and endoderm. An enucleated egg just after fertilization may also be used. Thus, virtually any type of cell may be isolated from the teratoma or by cells from the teratoma to developmentally differentiate. Specific cell markers unique to the particular cell type of interest are known in the art and may be used to identify the cloned primary cell.

In general, methods of making somatic cells of a different type than the cell used for nuclear transfer comprise:
a. transferring a first cell, the nucleus from said first cell, or the chromosomes from a first cell to a recipient oocyte or egg or other suitable recipient cell in order to generate an embryo;
b. obtaining an embryo having at least one cell, an inner cell mass, an embryonic disc and/or stem cell using said embryo;
c. injecting said inner cell mass, embryonic disc and/or stem cell into an immune compromised animal tissue culture or avian egg to form a teratoma;
d. isolating said resulting teratoma;
e. separating the different germ layers for the purpose of identifying specific cell types;
f. isolating a cell of a different type than the first cell.

In embodiments wherein the donor cell, nucleus or chromosomes are human, the genome of the primary cell may be modified such that the cell is incapable of producing a viable embryo. This may be affected by inactivating or knocking out one or more genes required for the formation of one of the three germ layers, or by expressing a “suicide” gene from a developmentally regulated promoter specifically expressed in a cell type contained in a germ layer which is not of interest Alternatively, gene knockouts or suicide gene expression could be targeted to genes specifically required for attachment to or development in a mammalian uterus.

As discussed above, preferably the first (nuclear donor) cell is a fibroblast. The method may be formed using any species of cell, and finds particular use in human therapeutic cloning in the generation of cloned organs and tissues for transplantation. Thus, the methods may be performed using human cells, and the primary cells isolated may be used to generate a tissue (for transplantation into a patient in need of a transplant).

Preferred types of primary cells to be generated by the disclosed methods are neurons, skeletal myoblasts, cardiac muscle, skin pancreatic β cells, endothelial cells, hematopoietic cells, skin cells, hair follicle cells, kidney cells and nerve cells. The method may further comprise isolating cells from the teratoma and growing said cells in the presence of growth factors to facilitate further differentiation. In particular, the genome of the first cell is altered prior to nuclear transfer, such that the new primary cells and engineered tissues that are generated express at least one therapeutic protein, or fail to express a native protein that may have been detrimental to the donor patient. The cells and tissues generated by the disclosed methods are also encompassed.

Preferred applications of cells and tissues generated by the methods disclosed herein include the production of neurons, pancreatic islet cells, hepatocytes, cardiomyocytes, hematopoietic cells, and other desired differentiated cell types and tissues containing.

These cells and tissues, which optionally may be transgenic, may be used for cell, tissue and organ transplantation, e.g., treatment of burns, hair transplantation, cancer, chronic pain, diabetes, dwarfism, epilepsy, heart disease such as myocardial infarction, hemophilic, infertility, kidney disease, liver disease, osteoarthritis, osteoporosis, stroke, affective disorders, Alzheimer's disease, enzymatic defects, Huntington's disease, hypocholesterolemine, hypoparathyroidase, immunodeficiencies, Lou Gehrig's disease, macular degeneration, multiple sclerosis, muscular dystrophy, Parkinson's disease, rheumatoid arthritis, spinal cord injuries and other trauma.

Because nuclear transfer techniques are useful in generating cloned mammals as well as cloned cells and tissues, the methods of the present invention are also useful in making cloned mammals having complex or compound genetic alterations. In addition the present invention is useful in producing animals that are young or more preferably hyper-young. In particular, the invention encompasses a method of re-cloning a cloned animal, wherein said re-cloned animal has been genetically altered with respect to the cloned animal. Such a method would not have been attempted without the finding of the present invention, which reveals that nuclear transfer rejuvenates late passage cells and restores telomere length. If the re-cloned mammal was of the same genetic age as the cloned genetic mammal (which is, in turn, the same genetic age of the first nuclear donor), the feasibility of the method would decline depending on the generation of the clone. The results obtained by the present inventors to-date suggest that this is not the case and that in fact re-cloning can be effectuated as many times as desired, and will result in “hyper-young” animals, embryos and cells. Hyper young typically animals have enhanced immune systems useful for generating antibodies, and improved coat pigmentation.

A preferred method of re-cloning according to the present invention comprises the following steps, and may be used to make a cloned animal having at least two genetic modifications:
a. obtaining a primary cell from an animal of interest,
b. making a first genetic modification to said primary cell by inserting heterologous DNA and/or deleting native DNA,
c. using said first genetically modified primary cell as a nuclear donor for nuclear transfer to an enucleated oocyte or egg or other suitable recipient cell,
d. obtaining a cloned embryo, fetus or animal having said first genetic modification,
e. obtaining a cloned primary cell from said cloned embryo, fetus or animal,
f. making a second genetic modification to said cloned primary cell by inserting heterologous DNA and/or deleting native DNA,
g. using said cloned primary cell having said first and second genetic modifications as a nuclear donor for nuclear transfer to an enucleated oocyte or egg or other suitable recipient cell,
h. obtaining a re-cloned embryo, fetus or animal having said first and second genetic modifications.

This process can be repeated as many times as desired. Preferably, at least one recloning step utilizes a donor cell that has been propagated to senescence or near-senescence, or checkpoint arrested, such that the telomeres of the reclones are regenerated or restored upon nuclear transfer. In particular, the method of the invention further comprises steps where said re-cloned embryo, fetus or animal is again re-cloned, and wherein a third genetic modification is made such that the further re-clone has the first, second and third genetic modifications. Accordingly, the method may be used to generate animals having numerous genes knocked out, inserted or substituted, and may be used to generate animals having entire cell systems replaced or modified, i.e., substitution of the human immunological system for that of the bovine, substitution of genes involved in complex enzymatic pathways such as those involving the clotting factors, or the complement cascade, etc.

The method of re-cloning of the present invention will allow the creation of complex animal models for the study of diseases which involve multiple genes and or cell types, and may not be able to be duplicated by the typical animal model which expresses a single transgene, or has a single gene of interest knocked out. Moreover, such animal models may be used to study the effect of therapeutic genes in a particular complex genetic background. Such animal models may also be used to produce and test products that regulate the expression of different genes, to knock out genes that are involved in eliciting immune responses, to substitute collagen genes or other structural proteins genes with homologous counterparts, etc.

The present invention involves the surprising discovery that senescent cells may be rejuvenated, that EPC-1 activity and other cell markers associated with aging may be increased, that telomorase may be activated and that telomeres are extended, and that tandem repeats are repaired, all by the process of nuclear transfer. Thus, the present invention involves the discovery of a new way to activate telomerase activity and/or EPC-1 activity, which has applications far beyond that of extending telomeres and replicative life-span. Also, we predict other repairs to tandemly repeated DNA sequences. In particular, the invention provides a method for isolating the mechanism(s) of telomerase activation, EPC-1 activation, or other aging related genes, as well as a means of regulating telomerase or EPC-1, or other genes using the identified mechanisms.

For instance, the cytoplasm of an oocyte can be fractionated and the fractions placed in association with a mortal cell, or a mortal cell nucleus, or telomeres, to assay for telomerase activation, EPC-1 activation (or other cell markers associated with aging) and telomere extension. Through such an assay, the active substituent or substituents in oocytes responsible for reactivating telomerase, EPC-1 activity, and/or other age associated cell markers can be identified and isolated. Similarly, RNA or cDNAs can be isolated from the oocyte and transfected into a mortal cell, or expressed in a cell-free system for detecting telomerase activity, and transfected cells or cell-free systems demonstrating telomerase activity may be identified. Such methods could be supplemented with subtractive hybridization techniques in order to enrich for RNAs which are expressed during embryogenesis and not during senescence. In this way, genes encoding enzymes potentially involved in telomerase activation may be identified.

Oocytes or eggs in the period just following fertilization may contain more than one gene or protein involved in telomerase activation. While not wishing to be held to any specific theory, the present inventors believe that there exists at least one regulatory protein or RNA in oocytes, or in ES cells or germ cells resulting from the development of oocytes, that is involved in the regulation of telomerase activity, and the ALT pathway and responds particularly to some aspect of the senescence cellular environment. It is possible that such protein(s) or RNA(s) activate telomerase or telomerase gene expression directly, but it is also possible that such proteins or RNAs work by inhibiting a suppressor of telomerase or the ALT pathway that exists or is expressed in senescent or near-senescent cells. A possible activator of telomerase is Oct 4 or Rex.

For instance, Xu et al. demonstrated that re-expression of the retinoblastoma protein in tumor cells induces senescence and inhibits telomerase activity (Oncogene (1997) 15:2589-2596). A recent report also suggests that a gene on chromosome 3 may be involved in transcriptional repression of hTERT, the catalytic subunit of telomerase. See http://claim.springer.de/EncRef/CancerResearch/samples/0001.htm. Several proteins have also been identified that interact directly with telomerase, such as p23/hsp90 (molecular chaperones) and TEPI (telomerase associated protein 1). Id. Researchers at Lawrence Berkeley National Laboratory have purported cloned two additional human telomere-associated proteins (Tin 1 and Tin 2). Federal Technology Report, Dec. 30, 1999, Partnership Digest, Technology Watch, p. 9. Thus, the regulatory mechanism identified by the present methods could operate by binding to or inhibiting the expression of a telomerase binding protein or a telomerase repressor, consequently increasing telomerase activity, but could also regulate telomerase activity by upregulating gene expression or enhancing protein stability.

The present invention includes methods of identifying at least one gene that either directly or indirectly enhances telomerase activity or the ALT pathway. Such methods could involve screening a cDNA or mRNA library generated from an embryo or embryonic stem cell for members that enhance telomerase or ALT activity in a senescent or near-senescent cell. The methods may also involve identifying at least one gene that either directly or indirectly suppresses telomerase or ALT activity, comprising, screening a cDNA or mRNA library generated from a senescent or near-senescent cell for members that suppress telomerase activity in an embryonic stem cell. Telomerase activity may be measured by any one of several methods known in the art, including measurement of reporter gene expression, e.g., a hTRT gene or protein fusion. A preferred reporter molecule is green fluorescent protein (GFP). Telomerase activity may also be measured using the TRAPeze assay. Screening methods may be combined with other known methods for the purpose of increasing the effectiveness of the screening procedure, for instance, by subjecting cbNA or mRNA libraries to subtractive hybridization with a cDNA or mRNA library from a senescent cell prior to library screening if the test library is generated from an oocyte or an ES cell, or vice versa.

The present invention also encompasses methods of identifying a protein that enhances telomerase, youthful patterns of gene expression or ALT activity, comprising (a) collecting fractions from the cytoplasm of an oocyte, embryo, or embryonic stem cell, (b) adding them to a cell-free system designed from a senescent or near-senescent cell, and (c) measuring for changes in telomerase, youthful gene expression or ALT activity that result from exposure to specific oocyte or ES cell cytoplasmic fractions. Methods for screening for compounds that inhibit telomerase or youthful gene expression are also included, and would comprise exposing an embryonic stem cell generated by nuclear transfer techniques using a senescent or near-senescent donor cell to a compound to determine whether said compound inhibits telomerase, youthful gene expression or ALT activity.

Also, the invention involves producing cells that have been transfected with the youthful gene, or regulating sequences, preferably linked to a suitable marker and method of using such cells to identify compounds that upregulate youthful gene expression. These screens should identify compounds that will modulate cell proliferation or aging. It is hypothesized that several genes may play a role in regulating cell proliferation and cycling, including EPC-1, the gas genes (Ciccarelli et al, Mci. Cell. Bid 10(4):1525-1529 (1990), such as gas-2, -3, -5, -7, PI-3 kinase (Tresini et al, Cancer Res. 58(i):1-4 (1998), collagenase, tPA, in regulating cell proliferation and cycling.

Another screen is the modification of a somatic cell, more preferably an aged somatic cell, with a marker gene, i.e., GFP, where the marker gene is fused to or associated with a gene whose expression is altered with cellular aging, i.e., telomerase. The telomerase gene and/or promoter may be fused to the marker gene in a series of truncated forms and the marker constructs may then be transfected into senescent or near—senescent cells. Nuclear transfer may then be used to identify region in the telomerase gene or gene promoter or upstream region involved in activating telomerase expression upon nuclear transfer.

Further, the invention involves placing the EPC-1 and other youthful genes under the control of a heterologous, e.g., regulatable and preferably strong promoter, and assessing the effect of increases or decreases in expression on telomerase activity and telomeres.

The present invention also includes the case of genetically modified somatic cells to identify the roles of genes in telomere regulation and the ALT pathway. Genes critical to ALT function can for instance be identified by the loss of ALT function when those genes are knocked out in the somatic cell prior to ALT.

The present invention also includes the regulatory compounds, proteins and nucleic acids identified by the methods described above and pharmaceutical compositions comprising the same, which may be isolated and employed as exogenous telomerase activating agents according to the methods and purposes described herein, i.e., for the treatment of age-related diseases, the treatment of aged tissues such as retinal cells, the therapy of cancer, and the improving the effectiveness of bone marrow transplants.

The scope and spirit of the present invention are illustrated by the way of the disclosed examples.

### EXAMPLE 1

### Fetal Donor Cells

This preliminary experiment suggested that somatic cell nuclear transfer can be used to restore the life-span of primary cultured cells. When fibroblasts from a six week-old fetus were cultured to senescence, they underwent approximately thirty population doublings, with an average cell cycle length of 28 to 30 hours. To test whether these cells could be rescued from senescence by nuclear transfer, a 40-day old fetus was generated using cells within 0.8 populations doublings from senescence. Fibroblasts derived from this fetus underwent 31 population doublings, as compared to 33 doublings for fibroblasts from a same-age fetus conceived normally. This data suggested that nuclear transfer is capable of rejuvenating senescent cells.

### EXAMPLE 2

### Cloned Calves Derived from Senescent Donor Somatic Cells

A somatic cell strain was derived from a 45-day-old female bovine fetus (BFF) and transfected with a PGK driven selection cassette. Cells were selected with G418 for 10 days, and five neomycin resistant colonies were isolated and analyzed for stable transfection by Southern blotting using a full length cDNA probe. One cell strain (CL53) was identified as 63% [total nuclei] positive for the transgene by FISH analysis, and was chosen for the nuclear transfer studies described in this study.

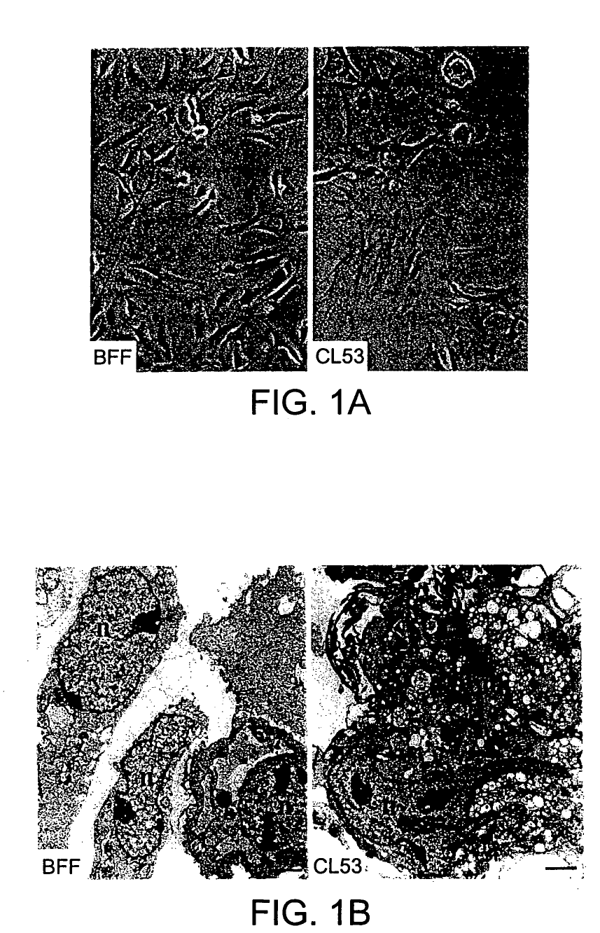

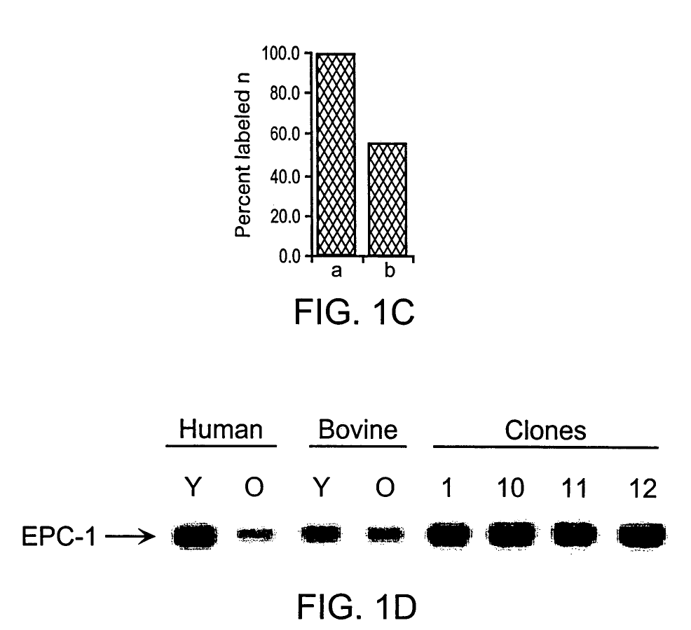

The CL53 fibroblast cells, which were characterized as negative for cytokeratin and positive for vimentin, were passaged until greater than 95% of their life-span was completed. The morphology of the cells was consistent with cells close to the end of their life-span as indicated by the phase contrast pictures of the cells by light microscopy (FIG. 1A). A more detailed ultrastructural analysis by electron microscopy demonstrated that these cells exhibited additional features of replicative senescence, including prominent and active Golgi apparati, increased invaginated and lobed nuclei, large lysosomal bodies, and an increase in cytoplasmic microfibrils as compared to the young cells (FIG. 1B) (27). In addition, these late passage cells exhibited a senescent phenotype in showing a reduced capacity to enter S phase as measured by a decrease in the incorporation of ³H-thymidine (FIG. 1C) and a significant increase in the staining of senescence-associated P-galactosidase (SA-β-gal; data not shown) (28). Furthermore, these cells exhibit a reduction in EPC-1 (early population doubling level cDNA-1)(29) mRNA levels as compared to early passage bovine BEF cells in a manner analogous to the changes observed during the aging of WI-38 cells (FIG. 1D).

A total of 1896 bovine oocytes were reconstructed by nuclear transfer using senescent CL53 cells as previously described (13). Eighty-seven blastocysts (5%) were identified after a week in culture. The majority of the embryos (n=79) were transferred into progestin-synchronized recipients, and 17 of the 32 recipients (53%) were detected pregnant by ultrasound 40 days after transfer. One fetus was electively removed at week 7 of gestation (ACT99-002), whereas 9 of the remaining recipients (29%) remained pregnant by 12 weeks of gestation. Three of these cows aborted at days 252 (twins), 253, and 278 of gestation. The remaining six recipients continued development to term. The rates of blastocyst formation (5%), and early (53%) and term (19%) pregnancies using senescent CL53 cells were comparable to those of control embryos produced using non-senescent donor (CL57) cells obtained from early passage BFF cells (5%, 45%, and 13%, respectively).

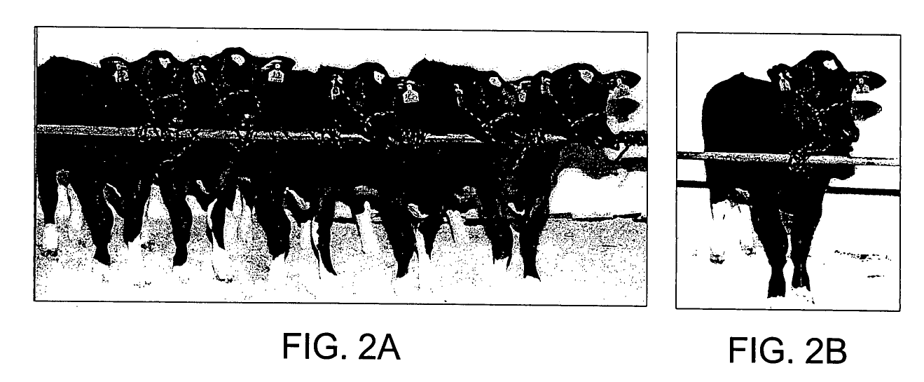

Calves CL53-1, CL53-8, CL53-9, CLS3-10, CL53-11, and CL53-12 were delivered by elective cesarean section at 280, 273, 273, 273, 266, and 266 days of gestation, respectively (FIG. 2). Genomic analyses confirmed the presence of the transgene in two of the animals (CL53-1 and CL53-12), as well as the fetus that was removed electively at day 49 of gestation. At birth, the presentation of the cloned calves was consistent with previous published reports (13, 15, 30, 31). In general, birth weights (51.6±3.6 kg) were. increased and several of the calves experienced pulmonary hypertension and respiratory distress at birth as well as incidence of fever after vaccinations at 4 months. Following the first 24 hours, the calves have been vigorous with minimal health problems. However, we have noted a moderate incidence of polyuria/polydypsia and lowered dry matter intake during the first two months. The occurrence of these complications was linked neither to the donor cell population (isolate 53 or 57) nor the presence or absence of transgene integration. After approximately two months all of the calves have performed well and resemble healthy control calves generated from both in vitro fertilization and in vivo embryo transfers. All six of the cloned animals remain alive and normal five to ten months after birth.

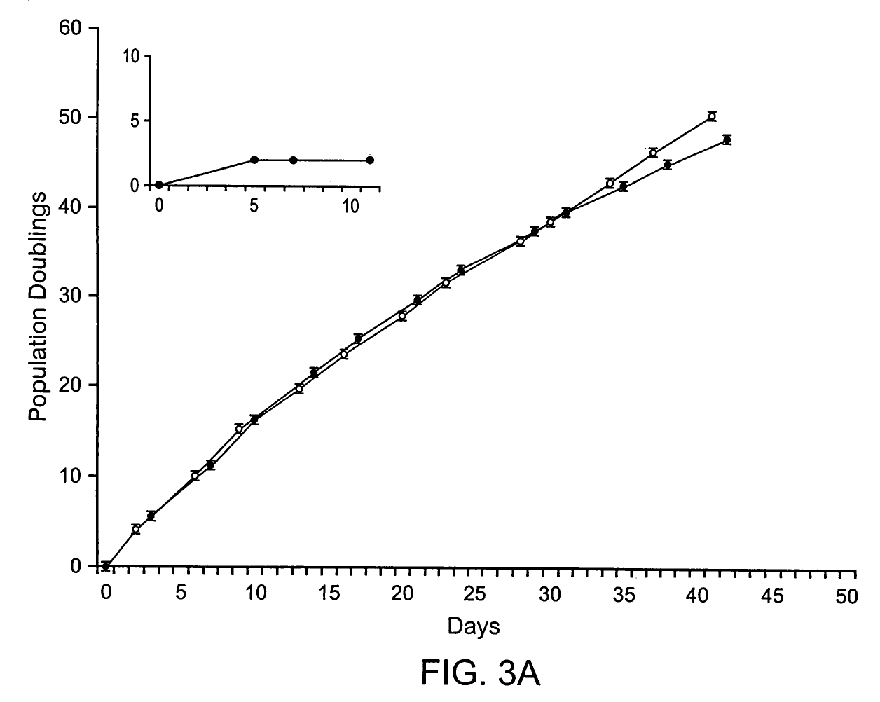

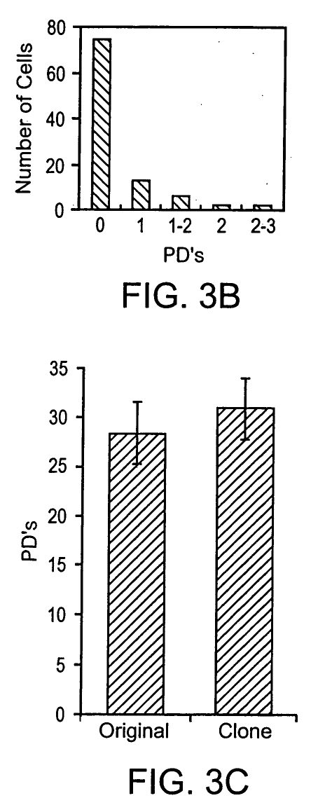

Dermal fibroblasts were isolated from the cloned calves, and mRNA prepared as described in FIG. 1D. The cells expressed EPC-1 mRNA levels comparable or higher than the early passage fetal cells. To exclude the possibility that there was a small proportion of nonsenescent cells that gave rise to the cloned animals, CL53 donor cells were seeded at both normal and clonal densities. As shown in FIG. 3B, the cells were 2.01±0.11 (SEM) population doublings from replicative senescence. Less than 12% (11/97) and 3% (2/97) of cells seeded at clonal density underwent more than 1 or 2 population doublings, respectively, whereas none of the cells divided more than 3 times (FIG. 3C). In contrast, early passage (pre-transfection) BFF cells underwent 47.8±0.9 population doublings, with an average cell cycle length of 17.8±0.7 hours during the logarithmic growth phase (FIG. 3A).

To test whether the somatic cell NT procedure restored the proliferative life-span of the senescent donor cells, we cultured fibroblasts from an electively removed 7-week-old fetus (ACT99-002). Cell strains from it underwent 85.3±5.6 population doublings, with a cell cycle length of 17.7±0.8 hours during the logarithmic growth phase (FIG. 3A). One-cell clones (n=5) were generated from the cloned (ACT99-002) and original (BFF) age-matched fetuses, and cultures characterized as fibroblasts by immunohistochemical staining were isolated. These one-cell clones underwent 31.2±3.4 and 25.9±2.9 population doublings from the cloned and original fetuses, respectively (FIG. 3D). These data suggest that cloning is capable of resetting the life-span of senescent cells, and that the cellular age of the fetus does not reflect the number of times the donor cells doubled in culture before NT.

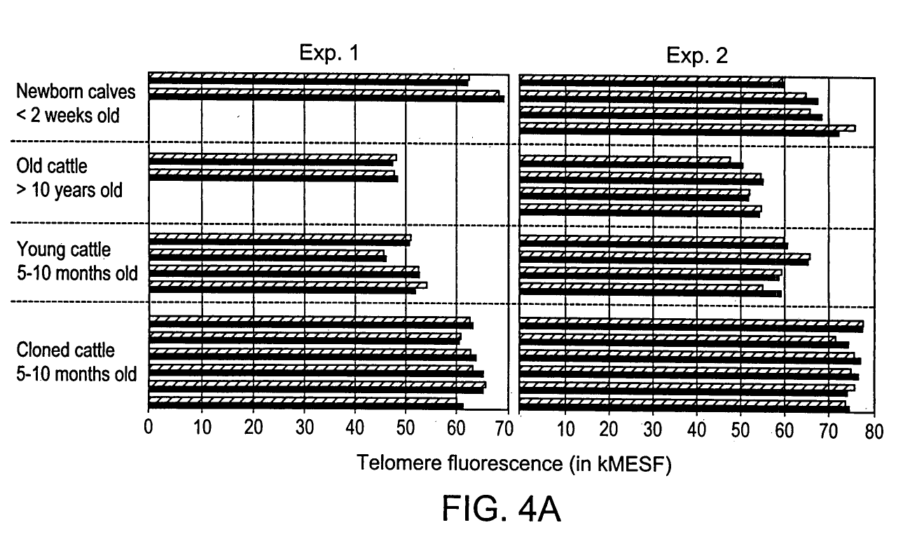

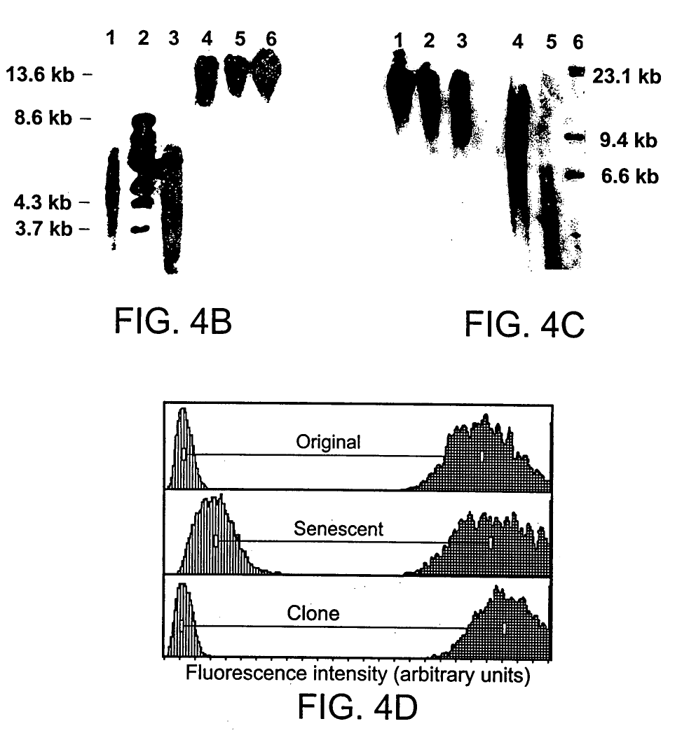

To further investigate the ability of NT to rescue senescent cells, the telomere lengths in nucleated blood cells of the cloned animals were compared to age-matched control animals, newborn calves (<2 weeks old) and old cows (10 to 19 years old) using flow cytometric analysis following in situ hybridization with directly FITC-labeled (CCCTAA) peptide nucleic acid probe (flow FISH) (32,33). The results of two separate experiments (FIG. 4A) are, indicative of complete restoration of telomere length (63.4±1.7 vs. 51.0±3.1 kMESF [mean±s.d., P<0.0001, exp. 1], and 75.7±1.7 vs. 61.4±3.2 kMESF [P<0.0001, exp. 2] in cloned animals relative to age-matched controls. Indeed, the telomeres of the clones animals were statistically longer than the four newborn calves (exp. 2) (75.3±1.2 vs. 66.9±1.4, P<0.0002). The mean telomere lengths of the old cattle were 47.7±0.7 kMESF and 52.0±3.6 kMESF for experiments 1 and 2, respectively.

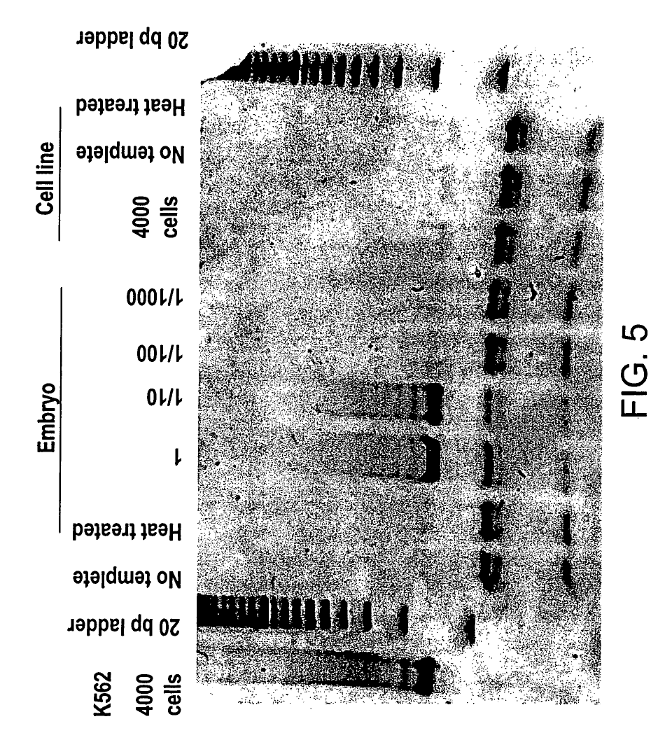

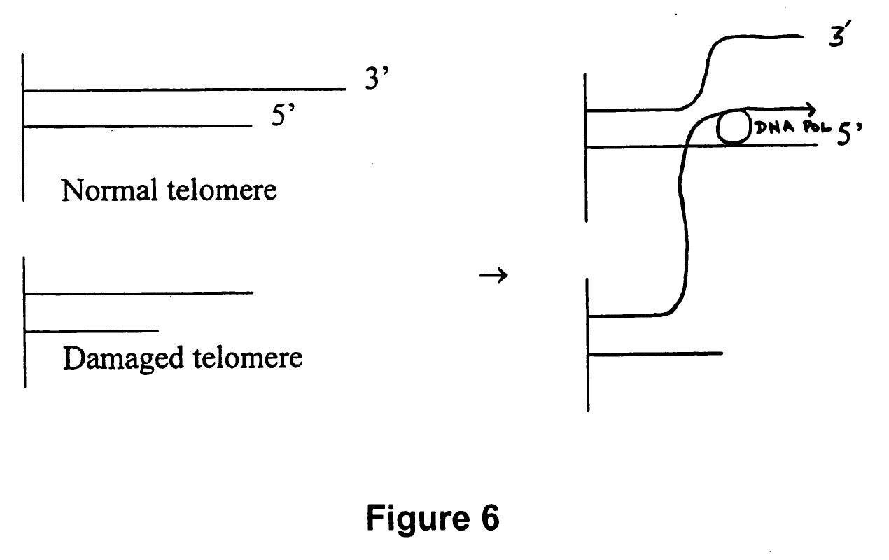

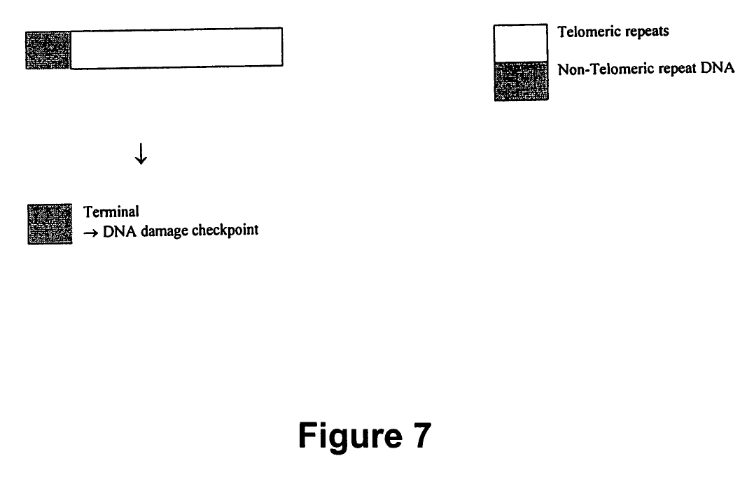

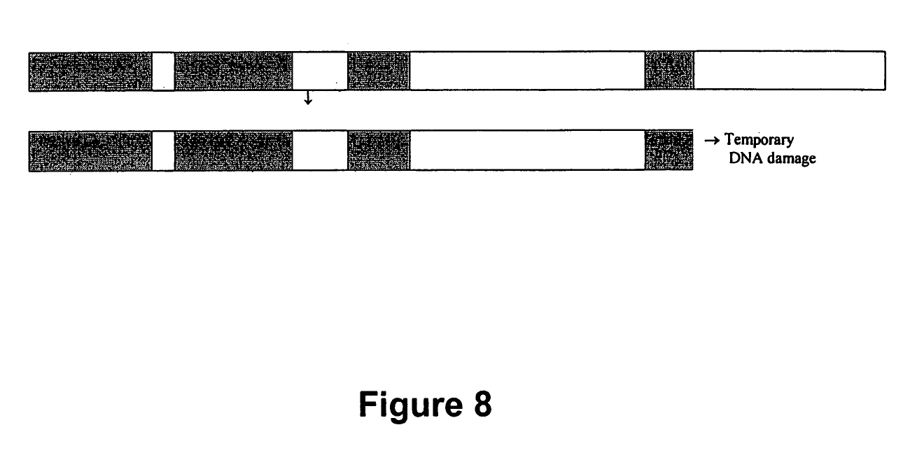

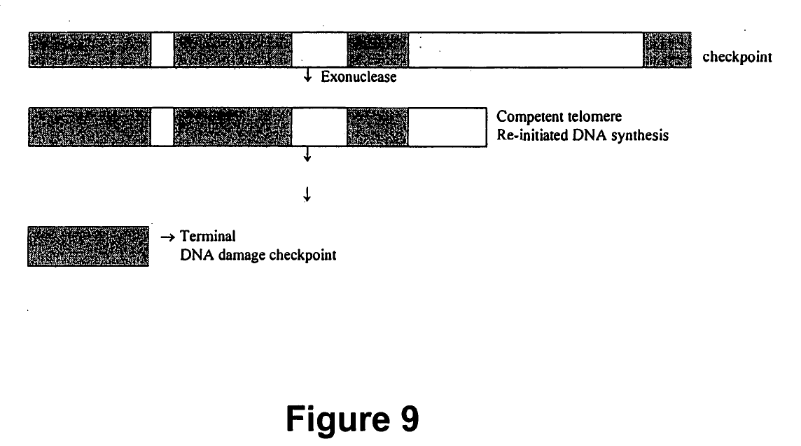

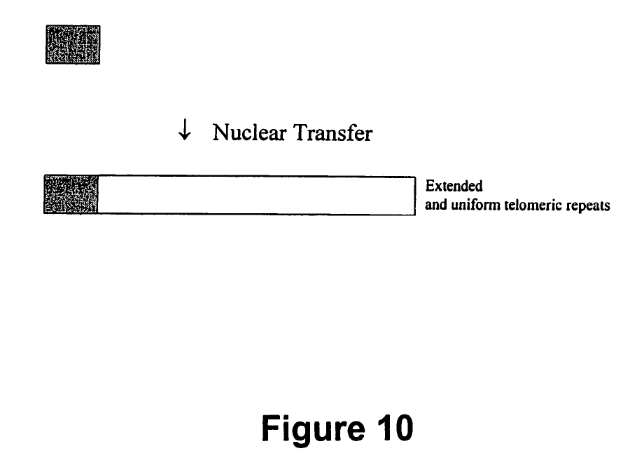

Telomere length dynamics was also studied in the senescent (CL53), control (pre-transfection BFF) and cloned (ACT99-002) cells using Southern analysis of terminal restriction fragments (34). The results (FIG. 4B-D) were consistent with the flow FISH analysis of the nucleated blood cells. The telomeres were longer in the cells derived from the cloned embryo (19.3 kb) than in the senescent and early-passage donor cells (16.2 and 17.9 kb, respectively) (compare lanes 4, 5 and 6, FIG. 4B). These results were confirmed by flow cytometric analysis of telomere length (flow FISH, ref 32) of the same cells (FIG. 4D). High levels of telomerase activity were also detected in reconstructed day 7 embryos tested by the TRAP assay (FIG. 5, lanes 5-8), whereas the bovine fibroblasts used as donor cells in the nuclear transfer experiments were negative (FIG. 5, lane 9).

Discussion

Telomere restoration has not been previously described in cloned animals. Our results differ markedly from the study by Shiels et al. (20), in which telomere erosion did not appear to be repaired after nuclear transfer in sheep. The telomere lengths of three cloned animals 6LL3 (Dolly, obtained from an adult donor cell), 6LL6 (derived from an embryonic donor cell) and 6LL7 (derived from a fetal donor cell) were found to be decreased relative to age-matched control animals. The authors suggested that full restoration of telomere length did not occur because these animals were generated without germline involvement. They further suggested that the shorter TRF in Dolly was consistent the time the donor cells spent in culture before nuclear transfer. The present findings are significant, not only because viable offspring were produced from senescent somatic cells, but because the nuclear transfer procedure appeared to extend the telomeres of the animals beyond that of newborn and age-matched control animals. It is not known whether the longevity of these animals will be reflected by the telomeric measurements, although cells derived from a cloned fetus were observed to have a longer proliferative life-span than those obtained from the original same-age nonmanipulated fetus. Indeed, the mean TRF size observed in the later cells was in agreement with these findings.

In discussions about cloning, it is commonly asked whether the animals generated by nuclear transfer are the result of the use of some rare cell rather than the majority of the cells in the culture. Mass cultures have multiple lineage's with various maximum achievable cell life-spans (43). Indeed, the late passage cells used in the present study represent cells that originally had the greatest life-span. If there were a subset of young cells with 20 or more population doublings remaining in the late passage culture, they would have out-proliferated the culture as is seen in mouse cell culture where spontaneous immortalization is common. In anticipation of this objection, we plated the donor cells at clonal densities and scored the proliferative life-span of every cell. Three-hundred and thirty-nine of the 347 cells (98%) underwent less than 3 PDs, whereas 347/347 (100%) underwent 4 or less PDs. Furthermore, the cells were grown in high serum (15%) concentrations, and young cells would have been rapidly proliferating and easily observed in the dish. The probability of a young cell in out sample is therefore <1/347. Seven animals (6 term animals and 1 fetus) were nevertheless cloned from the population of senescent fetal cells. It is therefore highly improbable that we, by chance, cloned the animals from undetectable young cells (P<0.001, Chi-square).

The differences between this study and that reported by Shiels et al. (20) could be due to differences in the choice of donor somatic cell types. Wilmut et al. (12), for instance, used quiescent (G₀) donor mammary epithelial cells to produce Dolly, whereas senescent (G₁) fibroblasts were used in the present experiments. Indeed, recent studies have shown that reconstruction of telomerase activity leads to telomere elongation and immortalization of normal human fibroblasts (35,36), whereas similar experiments using mammary epithelial cells did not result in elongation of telomeres and extended replicative life-span (37). Differences between cells in the ability of telomerase to extend telomeres, or in the signaling pathways activated upon adaptation to culture, were proposed to explain the differences (38). Other investigators, however, report that the exogenous expression of hTERT extends telomeres and immortalizes human mammary epithelial cells (J. Shay, personal communication).

Previous studies have documented significant up-regulation of telomerase activity during early bovine embryogenesis (39). The elongation of telomeres in the present study suggests that bovine embryos reconstructed by nuclear transfer contain a mechanism for telomere length regeneration and maintenance, providing chromosomal stability throughout the events of pre- and post-attachment development.

### EXAMPLE 3

### Nuclear Transfer Using Adult Donor Cells

The above data obtained with fetal fibroblast donors are consistent with experiments performed using senescent cells obtained from adult animals. Dermal fibroblasts were grown from three Holstein steers. Single cell clones were isolated and population doublings counted until senescence. Nuclear transfer was performed using these fibroblast cells that were at or near senescence. Fetuses were removed from the uterus at week 6 of gestation and fibroblasts isolated from them and cultured until senescence. Cells were analyzed by imunohistochemistry and were shown to be fibroblasts. The number of population doublings in the original cells from the adult animals at the time of nuclear transfer (counted as number of PDs before senescence) and from 6-week-old fetuses generated from them are shown in Table 1. Cell strains isolated from the cloned fetuses underwent an average of 89.4±0.9 PDs as compared to 60.5±1.7 PDs for cell strains generated from normal age-matched (6-week-old) control fetuses (P<0.0001). These data suggest that cloning is capable of resetting (and indeed, extending) the life-span of somatic cells, and that the cellular age of the fetus does not reflect the number of times the donor cells doubled in culture before NT.

TABLE 1
Population doublings in fibroblasts derived from
normal fetuses and fetuses generated from clonal
populations of adult senescent cells

| | PDs left at time of nuclear | PDs in fibroblasts |
|---|---|---|
| | transfer in original adult cells | isolated from the fetus |
| Cloned Fetus | | |
| 25-1 | 0.26 | 90.14 |
| 25-2 | 0.0 | 91.44 |
| 14-1 | 4.0 | 89.27 |
| 14-2 | 1.0 | 90.34 |
| 22-1 | 2.5 | 85.86 |
| Normal fetus | | |
| 1-1 | — | 59.64 |
| 2-1 | — | 67.37 |
| 3-1 | — | 60.18 |
| 3-2 | — | 59.82 |
| 3-3 | — | 55.66 |

### EXAMPLE 4

### Analysis of Adult Donor Cell Types

Tissue biopsies will be obtained from all three germ layers from an adult cow (obtained at time of slaughter). In particular at least the following cells will be collected:
ectoderm-keratinocytes
mesoderm-dermal fibroblasts
endoderm-gut epithelium

A portion of the above three cell types will immediately be evaluated to determine telomere length. This can be affected by various methods. The remaining portion of all three cell types will be cultured until senescence. During culturing, a portion of each population will be retained and frozen. The different frozen cell samples will be labeled based on their particular population doubling.

Thereafter, the telomere length for the various cell samples will be evaluated, including especially the cells obtained at the time of senescence.

### EXAMPLE 5

### Cloned Calves Generated from Adult Senescent Donor Somatic Cells

The cells obtained from Example 4 will be used to obtain cloned bovine fetuses. In particular, bovine clones will be produced using all 3 cell types, and using cells from different population doublings, i.e., from 0.8 population doublings away from senescence. The cloned bovine fetuses will be produced substantially according to the methods disclosed in U.S. Pat. No. 5,945,577, incorporated by reference herein. The cloned fetuses will be removed at forty days and cells of all three types isolated therefrom, e.g., keratinocytes, dermal fibroblasts, and gut epithelial cells.

Additionally, as a control, two same-age (40 day) wild-type fetuses will also be used to recover the same three types of cells. These cells, as well as those isolated from the cloned fetuses, will be cultured until senescence.

Again, telomere length of these different types of cultured cells will be determined immediately upon isolation from the animal or from such cells which are frozen upon isolation. Further, cells will again be removed and frozen from different cell populations until senescence. Thereafter, telomere length will be computed for the different cell types obtained at different cell population doublings, for cultured cells derived from cloned and wild-type embryos.

The results will be compared to the results of Example 4. These experiments are currently ongoing.

### EXAMPLE 6

### EPC-1 Expression in Young Vs. Old Cells

EPC-1 expression was compared in human, bovine cells that were young or old, in cloned animals, and in controls. These results are shown below.

These results suggest that young cells from cloned animals are younger than young cells obtained from normal animals as measured by EPC-1 expression. Telomere length as a marker was also restored to younger levels in these cells. The explanation for this may be that telomeres have an imperfect nature while maintained at long lengths in the immortal given line, telomerase does not have frequent access to internal sequences. Therefore, the (T₂AG₃)ₙ repeats break down in fidelity as one reads the sequences from the telomere going toward the centromere. This is shown schematically below.

Senescence minor problems have been repaired by 3′ □5′ exonuclease that then again expose T₂AG₃ which restores binding to TRF-2. However, at some point the damage is so substantial it triggers what is known as terminal cell senescence.

Wilmut argued that cloning from a senescent cell may lead to problems in animals because telomere length reflects the shortened telomere of the somatic cell donor nucleus. However, our results suggest the exact reverse. Rather, growing a cell to senesence or near-senescence, or checkpoint arrested, allowing the cell to lose T₂AG₃, removing minor damage along the way by 3′ 0 5′ exonuclease may afford an opportunity to then transfer that gene into an enucleated oocyte or other embryonic cell and with a subsequent burst of telomerase activity to rebuild a tract of pure T₂AG₃ (longer than normally present). These cells will possess longer life-spans, but also, because of the purity of T₂AG₃ would rarely have cells in temporary cell cycle arrest. This would result in higher than normal mitotic cell index and overall a “younger than young” pattern of gene expansion.

To more thoroughly investigate this, experiments are being conducted using cultures from age-matched mammals and cloned samples, cloned from young and sensecent cells and those with or without shortened telomere. These cells are grown to senescence and frozen back every 15 pd. These cells will be compared with respect to marker of cell senescence. Gene expression will be compared in these cells by known methods, e.g. northern blots or by labeling using suitable probes.

### EXAMPLE 7

### Elevated Telomerase Levels in Embryos Derived from Nuclear Transfer

To investigate the mechanism of telomere extension, the levels of telomerase activity in early embryonic development following nuclear transfer (NT) were examined in the bovine system. Similar to results that were previously published for normal (IVF) bovine embryos (Betts and King, 1999, Dev. Genetics 25:397-403), telomerase was detectable in all of the stages of early development that were analyzed. Levels of telomerase activity decreased at the 8-16 cell sage, and then increased at the morula and blastocyst stages for both the NT and IVF control embryos (data not shown). However, the levels of telomerase in the NT blastocysts were 2-fold higher than corresponding IVF blastocysts.

### EXAMPLE 8

### Hyper-Young Immune Function in Cloned Animals Vs. Age-Matched Controls

To investigate the extent to which immune senescence is reversed upon nuclear transfer, and to determine whether cloned animals demonstrate enhanced immune function as compared to age-matched controls, the immune responses of cells from cloned vs. control cows following in vitro exposure to various mitogens was examined. There were significant differences between the cloned and control animals in response to TSST (toxic shock syndrome toxin, a bacterial superantigen that induces T cell proliferation), and in response to pokeweed mitogen (PWM), which induces both T and B cell proliferation. The differences were observed in both 2 day and 3 day cultures. Differences were also observed in response to PHA (another T cell mitogen), but with more variation. The significance of differences observed with PHA responses may be determined by testing a larger number of subjects. Differences in response to Con A (a T cell mitogen) were small and not statistically significant. For TSST and PWM, the differences are about 2-fold, with the 72 hr TSST system showing a 2.6× effect. Results are given in the table below.

In vivo responses to the mitogens were not tested given the heightened sensitivity of the cloned animals observed following vaccination. However, in vivo tests for skin delayed type hypersensitivity responses to recall antigens would pose a low risk and could be performed to analyze in vivo immune responses.

In vitro tests to examine the responses of specific cell types, ie., T cell subsets, B cells, macrophages, etc., may also be pursued using reagents useful for separating specific cell types. The levels of production of specific cytokinesis may also be examined using routine methods.

Mean Values for 78 hr cultures

| Cow | Group | None | Con_2 | Con_5 | PWM | PHA | TSST |
|---|---|---|---|---|---|---|---|
| C245 | Controls | 302 | 39038 | 35334 | 4435 | 3459 | 4011 |
| C246 | Controls | 142 | 27124 | 28010 | 7118 | 1188 | 6025 |
| C247 | Controls | 327 | 29154 | 38478 | 6555 | 2373 | 6945 |
| C248 | Controls | 512 | 30374 | 32072 | 8046 | 2972 | 9421 |
| C249 | Controls | 278 | 56841 | 49533 | 11016 | 11338 | 15039 |
| C250 | Controls | 147 | 29912 | 24270 | 11035 | 2334 | 13575 |
| Mean | | 285 | 35407 | 34616 | 8034 | 3944 | 9169 |
| SD | | 137 | 11277 | 8889 | 2603 | 3701 | 4367 |
| E1 | Expt1 | 422 | 64492 | 61135 | 16851 | 12479 | 22185 |
| E8 | Expt1 | 234 | 47037 | 43113 | 13496 | 7986 | 15199 |
| E9 | Expt1 | 472 | 31735 | 34609 | 9511 | 7130 | 11320 |
| E10 | Expt1 | 569 | 43051 | 36157 | 14647 | 10788 | 23357 |
| E11 | Expt1 | 148 | 49339 | 41446 | 21701 | 2572 | 44759 |
| E12 | Expt1 | 352 | 54187 | 45248 | 18829 | 8795 | 25907 |
| Mean | | 366 | 48307 | 43618 | 15841 | 8292 | 23788 |
| SD | | 155 | 10968 | 9501 | 4273 | 3411 | 11630 |
| p-value: | | 0.032 | 0.06 | 0.09 | 0.00 | 0.05 | 0.01 |
| Effect size: | | 29% | 36% | 26% | 97% | 110% | 159% |

### CONCLUSION AND BROAD APPLICATION OF INVENTION

As we disclose herein, the extension of telomeres in somatic cells by NT was itself nonobvious in light of Wilmut. But the fact that starting from a senescent cell would lead to even better results (longer telomeres, longer lived cells) is nonobvious even in light of the former result.

There is very little consensus even now as to the mechanisms that translate telomere shortening into the phenotype of cell senescence or the intermediate slowing of the cell cycle. Early papers proposed that the progressive loss of telomeric repeats led to the loss of telomeric genes and the loss of critical cell function. Woodring E. Wright proposed a model a few years ago that telomere shortening shifted the heterocbromatin associated with the telomere to silence a telomeric gene or genes that in turn led to senescence. Bryant Villeponteau published almost the opposite, that is, that there was a cone of heterochromatin associated with the telomere that shortened with telomere shortening and that this activated genes near the telomere. Titia de Lange in a recent paper on TRF2 and T loops proposed that senescent cells can no longer bind TRF2 and form T loops. However, another possibility is that “sprinkled” throughout the telomere are nontelomeric sequences where there are more tracts of pure TTAGGG at the very telomere and fewer internally. As telomeres shorten in somatic cells, the cells increasingly encounter nontelomeric sequences at the telomere that cannot bind TRF2 and eventually this raises the levels of activated p53 and then p21 to cause a slowing and eventual cessation of the cell cycle. The point is that this may not be an all or none phenomenon with a young cell proliferating and suddenly becoming senescent. It may be a gradient of increasing amount of damaged telomeres progressively raising p21.

Our results suggest that the artificial removal of telomeres through senescence and then the rapid resynthesis of accurate TTAGGG following NT, may lead to cells and animals that have the ability to proliferate in a younger state longer than a normal cell. There is no reason for evolution to select for cells or animals that would live longer than they need to reproduce. So there is no reason for the germ line to give the soma cells more uniform TTAGGG than they need. The technique of growing cells to senescence would effectively strip away the good and the bad telomeric sequences and then NT would give the cells a better longevity potential than they ever had normally. This would be the case even if the cells had comparable telomere length to those of normal cells. This would lead to cells that had a higher mitotic index for a longer period of time, and therefore animals that aged better and lived longer.

Therefore, the uses of the subject NT method with telomere extension, or even without telomere extension, may result in the resynthesis of new uniform TTAGGG in the telomeres. While not being bound by their hypothesis, the inventors believe that this may occur via the upregulation of telomerase, EPC-1, alone or in association with other genes such as growth arrest sequences (gas genes), collagenase, tPA, and others. This should result in longer lived and healthier animals, and cells for human therapy that are “hyper-youthful.”This is the first demonstration of hyper-youthful cells, that is, a population of cells and tissues with an overall phenotype that is, even more young than a normal mixed population of young cells, that is, a pattern of gene expression and mitotic index more youthful than normal youthful cells. The use of telomerase merely extended telomeres and the life-span of cells. However, to the inventors' knowledge, all of the published reports showed no evidence that cells could be obtained wherein the overall phenotype of such cells is younger or hyper-young. Indeed, many researchers report that old, but not yet senescent cells that have slowed down, continue to divide slowly, but indefinitely withe telomerase.

A preferred application of the invention would be to keep telomeres as short as would allow the desired life-span, but to maximize the uniformity of TTAGGG. This would optimize the delicate balance of longevity vs. cancer risk, that is, the cells would not be constitutively immortal, and they would not have longer telomeres than necessary so as to limit the clonal expansion of abnormal cells.

Animals cloned from senescent cells using this technology would be predicted to have unique properties. For example, animals raised for their coats, would be predicted to have more uniform coat color, would have an increased immune response would be more disease resistant, and have other advantages.

Specific medical applications could be, as we said, for age-related disease, such as age-related macular degeneration, Parkinson's, Alzheimer's, osteoartbritis, osteoporosis, immune senescence, skin aging, emphysema, aneurisms, coronary heart disease, hypertension, cataracts, adult onset diabetes, and so on. In addition, diseases associated with an accelerated cell turnover such as muscular dystrophy, herpes zoster, AIDS, and cirrhosis could be treated by administering regenerated cells.

1. A method of rejuvenating a primary cell, comprising:
a. transferring a primary cell, the nucleus from said primary cell or chromosomes from a primary cell to a recipient oocyte or egg in order to generate an embryo;
b. obtaining an inner cell mass, embryonic disc and/or stem cell using said embryo;
c. injecting said inner cell mass, embryonic disc and/or stem cell into an immune-compromised animal to form a teratoma;
d. isolating said resulting teratoma;
e. separating the different germ layers for the purpose of identifying specific cell types;
f. isolating a cell of the same type as the primary cell.

2-86. (canceled)
